(19) United States
(12) Patent Application Publication
(10) Pub. No.: US 2011/0105990 A1
(43) Pub. Date: May 5, 2011

(54) ZONAL DRUG DELIVERY DEVICE AND METHOD

(76) Inventor: Thomas A. Silvestrini, Alamo, CA (US)

(21) Appl. No.: 12/939,033

(22) Filed: Nov. 3, 2010

Related U.S. Application Data
(60) Provisional application No. 61/258,130, filed on Nov. 4, 2009.

(51) Int. Cl. A61F 9/007

(52) U.S. Cl. 604/8

(57) ABSTRACT

Disclosed herein are ocular implants for treating an eye and methods of implantation including an elongate member having a proximal end with at least one inflow port, a distal end with at least one outflow port, and a longitudinal, internal lumen extending through the elongate member. The distal end of the elongate member is in fluid communication with the suprachoroidal space such that the proximal end of the elongate member remains in fluid communication with the anterior chamber when the elongate member is implanted in the eye. At least one polymeric film surrounds at least a portion of the elongate member, the film comprising a first drug delivery zone embedded with a first drug, wherein the first drug diffuses from the polymeric film over time into the eye at a first anatomical location.

### REFERENCE TO PRIORITY DOCUMENT

This application claims priority of U.S. Provisional Patent Application Ser. No. 61/258,130, entitled “Zonal Drug Delivery Device and Method” by Thomas Silvestrini, filed Nov. 4, 2009. Priority of the filing date of Nov. 4, 2009 is hereby claimed, and the disclosure of the provisional patent application is hereby incorporated by reference in its entirety.

### BACKGROUND

This disclosure relates generally to methods and devices for use in treating glaucoma. In particular, this disclosure relates to implantable drug delivery devices that reduce aqueous humor production in the eye.

The mechanisms that cause glaucoma are not completely known. It is known that glaucoma results in abnormally high pressure in the eye, which leads to optic nerve damage. Over time, the increased pressure can cause damage to the optic nerve, which can lead to blindness. Treatment strategies have focused on keeping the intraocular pressure down in order to preserve as much vision as possible over the remainder of the patient's life.

Unfortunately, drug treatments and surgical treatments available still need much improvement, as they can cause adverse side effects and often fail to adequately control intraocular pressure.

### SUMMARY

The subject matter described herein provides many advantages. For example, the current subject matter includes improved devices and methods for the treatment of eye diseases such as glaucoma that are low profile, simple and use minimally-invasive delivery procedures. The implants described herein are designed to reduce aqueous humor production in the eye and improve outflow of aqueous humor from the anterior chamber.

In one aspect, an ocular implant is described that includes an elongate member having a proximal end with at least one inflow port, a distal end with at least one outflow port, and a longitudinal, internal lumen extending through the elongate member. The distal end of the elongate member is in fluid communication with the suprachoroidal space such that the proximal end of the elongate member remains in fluid communication with the anterior chamber when the elongate member is implanted in the eye. At least one polymeric film surrounds at least a portion of the elongate member. The film includes a first drug delivery zone embedded with a first drug, wherein the first drug diffuses from the polymeric film over time into the eye at a first anatomical location.

In another aspect, the ocular implants described herein can include a second drug delivery zone embedded with a second drug and the second drug diffuses from the polymeric film into the eye over time at a second anatomical location. The first anatomical location can include the ciliary body, epithelial cells of the ciliary body, the boundary between ciliary body and sclera, or the suprachoroidal space. The first drug can reduce aqueous humor production or inflow of aqueous humor to the anterior chamber. The first drug can be a carbonic anhydrase inhibitor, beta blocker, and an alpha-agonist or a combination thereof. The second anatomical location can be the anterior chamber, iris, or trabecular meshwork. The second drug can increase outflow of aqueous humor from the anterior chamber. The second drug can be a prostaglandin, prostaglandin analogue, muscarinic, pilocarpine, carbachol, alpha 2 adrenergic agonist, and epinephrine, or a combination thereof. The polymeric film can include a biocompatible material selected from the group consisting of poly(lactic acid), polyethylene-vinyl acetate, polybutyl methacrylic, poly(lactic-co-glycolic acid), poly(D,L-lactide), poly(D,L-lactide-co-trimethylene carbonate), collagen, heparinized collagen, poly(caprolactone), poly(glycolic acid), a copolymer and a combination thereof. The first drug and the second drug can or cannot be the same drug. The implant can further include a second polymeric film surrounding at least a portion of the first polymeric film. The second polymeric film can alter a parameter of diffusion kinetics of the first drug from the first drug delivery zone.

More details of the devices, systems and methods are set forth in the accompanying drawings and the description below. Other features and advantages will be apparent from the description and drawings.

### BRIEF DESCRIPTION OF THE DRAWINGS

These and other aspects will now be described in detail with reference to the following drawings. Generally speaking the figures are not to scale in absolute terms or comparatively but are intended to be illustrative. Also, relative placement of features and elements may be modified for the purpose of illustrative clarity

FIG. 1 is a cross-sectional, perspective view of a portion of the eye showing the anterior and posterior chambers of the eye.

FIG. 2 is a cross-sectional view of a human eye.

FIG. 3 shows an embodiment of an implant that reduces aqueous humor production.

FIG. 4 shows relative shapes of an implant and the suprachoroidal space.

FIG. 5 shows an implant positioned within the suprachoroidal space.

FIGS. 6A-6E show embodiments of drug delivery implants that reduce aqueous humor production.

### DETAILED DESCRIPTION

Described herein are devices, systems and methods for the treatment of eye diseases such as glaucoma. The devices described can deliver therapeutics to select regions and structures within the eye by the formulation of drug delivery zones in an implant as will be described in more detail below.

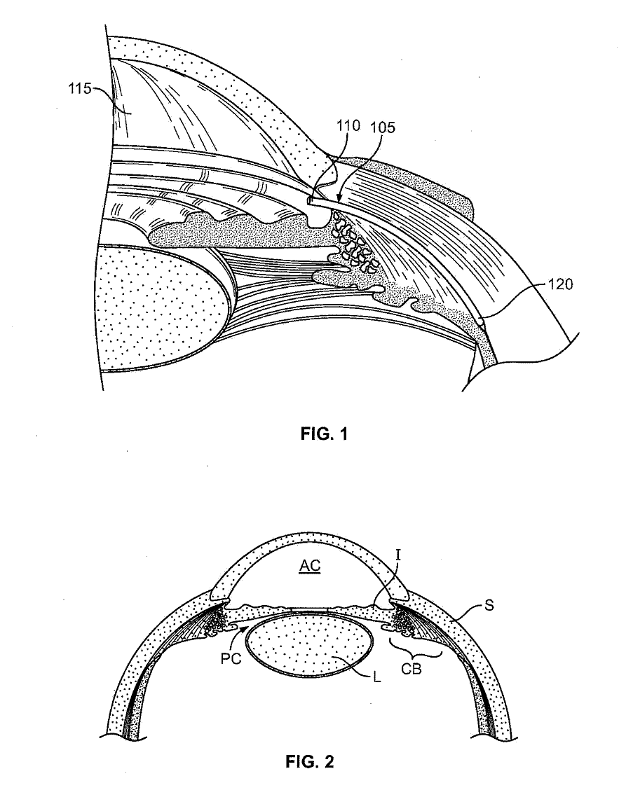

FIG. 1 is a cross-sectional, perspective view of a portion of the eye showing the anterior and posterior chambers of the eye. A schematic representation of an implant 105 is positioned inside the eye such that a proximal end 110 is located in the anterior chamber 115 and a distal end 120 is located in or near the suprachoroidal space (sometimes referred to as the perichoroidal space). The suprachoroidal space can include the region between the sclera and the choroid. The suprachoroidal space can also include the region between the sclera and the ciliary body. In this regard, the region of the suprachoroidal space between the sclera and the ciliary body may sometimes be referred to as the supraciliary space. The implants described herein are not necessarily positioned between the choroid and the sclera. The implants can be positioned at least partially between the ciliary body and the sclera or it can be at least partially positioned between the sclera and the choroid. In any event, the implants can provide a fluid pathway between the anterior chamber and the suprachoroidal space.

In an embodiment, the implant 105 can be an elongate element having one or more internal lumens through which aqueous humor can flow from the anterior chamber 115 into the suprachoroidal space. The implant 105 can have a substantially uniform diameter along its entire length, although the shape of the implant 105 can vary along its length (either before or after insertion of the implant), as described below. Moreover, the implant 105 can have various cross-sectional shapes (such as a, circular, oval or rectangular shape) and can vary in cross-sectional shape moving along its length. The cross-sectional shape can be selected to facilitate easy insertion into the eye. U.S. Patent Publication Nos. 2007-0191863 and 2009-0182421 describe exemplary implants. These applications are incorporated by reference in their entirety.

At least a portion of the implant can be formed of a structure having a stiffness that causes the implant 105 to form a chord (either straight or curved) relative to the curvature of the suprachoroidal space, as described in detail below. That is, the implant can define a line that intersects at least two points along a curve that conforms to the natural curvature of the suprachoroidal space if the implant were not present. The implant 105 can have a stiffness that is greater than the stiffness of adjacent eye tissue (e.g., the choroid and the sclera) such that the implant 105 deforms the eye tissue and forms a chord relative to the curvature of the suprachoroidal space when implanted in the eye. The presence of the implant 105 can cause the suprachoroidal space to achieve a geometry that produces a tented volume within the suprachoroidal space.

FIG. 2 is a cross-sectional view of a portion of the human eye. The eye is generally spherical and is covered on the outside by the sclera S. The retina lines the inside posterior half of the eye. The retina registers the light and sends signals to the brain via the optic nerve. The bulk of the eye is filled and supported by the vitreous body, a clear, jelly-like substance. The elastic lens L is located near the front of the eye. The lens L provides adjustment of focus and is suspended within a capsular bag from the ciliary body CB, which contains the muscles that change the focal length of the lens L. A volume in front of the lens L is divided into two by the iris I, which controls the aperture of the lens L and the amount of light striking the retina. The pupil is a hole in the center of the iris I through which light passes. The volume between the iris I and the lens L is the posterior chamber PC. The volume between the iris I and the cornea is the anterior chamber AC. Both chambers are filled with a clear liquid known as aqueous humor.

The ciliary body CB continuously forms aqueous humor in the posterior chamber PC by secretion from the blood vessels. The aqueous humor flows around the lens L and iris I into the anterior chamber AC and exits the eye through the trabecular meshwork, a sieve-like structure situated at the corner of the iris I and the wall of the eye (the corner is known as the iridocorneal angle). Some of the aqueous humor filters through the trabecular meshwork near the iris root into Schlemm's canal, a small channel that drains into the ocular veins. A smaller portion rejoins the venous circulation after passing through the ciliary body and eventually through the sclera (the uveoscleral route).

Glaucoma is a disease wherein the aqueous humor builds up within the eye. In a healthy eye, the ciliary processes secrete aqueous humor, which then passes through the angle between the cornea and the iris. Glaucoma appears to be the result of clogging in the trabecular meshwork. The clogging can be caused by the exfoliation of cells or other debris. When the aqueous humor does not drain properly from the clogged meshwork, it builds up and causes increased pressure in the eye, particularly on the blood vessels that lead to the optic nerve. The high pressure on the blood vessels can result in death of retinal ganglion cells and eventual blindness.

Closed angle (acute) glaucoma can occur in people who were born with a narrow angle between the iris and the cornea (the anterior chamber angle). This is more common in people who are farsighted (they see objects in the distance better than those which are close up). The iris can slip forward and suddenly close off the exit of aqueous humor, and a sudden increase in pressure within the eye follows.

Open angle (chronic) glaucoma is by far the most common type of glaucoma. In open angle glaucoma, the iris does not block the drainage angle as it does in acute glaucoma. Instead, the fluid outlet channels within the wall of the eye gradually narrow with time. The disease usually affects both eyes, and over a period of years the consistently elevated pressure slowly damages the optic nerve.

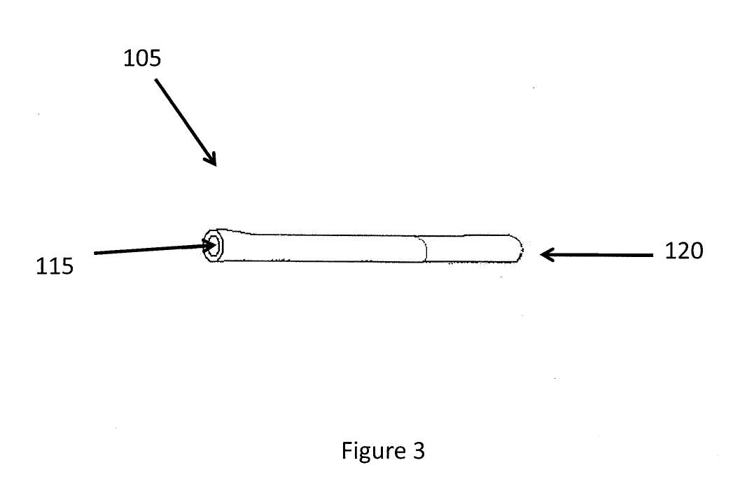

FIG. 3 shows a first embodiment of an implant 105. The implant 105 can be an elongate member having a proximal end, a distal end, and a structure that permits fluid (such as aqueous humor) to flow along the length of the implant such as through or around the implant from the anterior chamber to the suprachoroidal space. In the embodiment of FIG. 3, the implant 105 can include at least one internal lumen having at least one opening 115 for ingress of fluid (such as aqueous humor from the anterior chamber) into and at least one opening 120 for egrets of fluid (such as into the suprachoroidal space) from an internal lumen 110 (see FIG. 1).

The internal lumen 110 can serve as a passageway for the flow of aqueous humor through the implant 105 directly from the anterior chamber to the suprachoroidal space. The internal lumen 110 can also be used as a pathway for flowing irrigation fluid into the eye generally for flushing or to maintain pressure in the anterior chamber. In the embodiment of FIG. 3, the implant 105 can have a substantially uniform diameter along its entire length, although the shape of the implant 105 can vary along its length (either before or after insertion of the implant). Moreover, the implant 105 can have various cross-sectional shapes (such as a circular, oval or rectangular shape) and can vary in cross-sectional shape moving along its length. The cross-sectional shape can be selected to facilitate easy insertion into the eye.

The implant 105 can include any number of additional structural features that aid in anchoring or retaining the implanted implant 105 in the eye. The implant 105 can include one or more additional retaining or retention structures, such as protrusions, wings, tines, or prongs that lodge into anatomy to retain the implant in place.

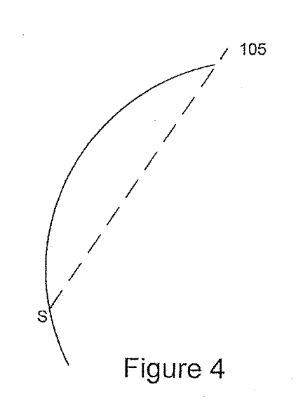

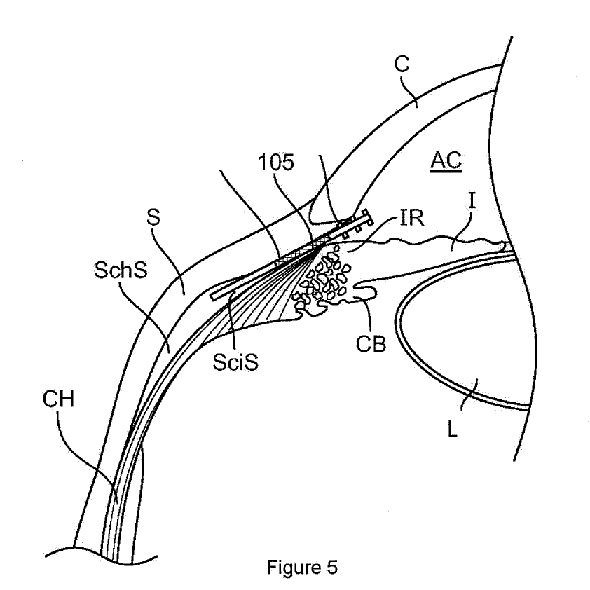

As illustrated schematically in FIG. 4, when implanted in the eye the implant 105 can form a dissection plane within or near the suprachoroidal space. The dissection plane can be straight or it can be curved as the dissection plane is being formed. At least a portion of the suprachoroidal space can be described as the space between two curved shells: a first, outer shell comprising the scleral tissue and a second, inner shell comprising the choroidal tissue. The shells can abut one another in that the inner surface of the sclera abuts the outer surface of the choroid with the suprachoroidal space being a virtual space that exists when the sclera is separated from the choroid. The sclera has a tougher texture than the choroid. The implant 105 can have a stiffness such that its presence in or near the suprachoroidal space can increase or decrease ratios of curvature of one or both of the shells by pushing against the tough outer shell and/or the fragile inner shell. If the dissection plane is curved, the dissection plane can have a curvature that will follow a dissecting wire that performs the dissection or that is governed by the shape and/or stiffness of the implant positioned in the dissection plane. As an alternative, the implant itself can perform the dissection. The curvature can be different from the curvature of the suprachoroidal space when the implant is implanted in the eye. Thus, the implant can form a straight or curved chord relative to the natural curvature of the suprachoroidal space if the implant were not present in the suprachoroidal space.

FIG. 4 shows a curve S (in solid line) that represents the natural curvature of the suprachoroidal space when the implant is not present. The implant 105 (represented by a dashed line) can be a straight implant or a curved implant that intersects the natural curvature S but does not conform to the natural curvature when implanted. The implant 105 can have a relative stiffness such that, when implanted, the implant 105 can deform at least a portion of the tissue adjacent the suprachoroidal space to take on a shape that is different than the natural curvature. In this manner, the implant 105 can form a tent or volume between the tissue boundaries (formed by the sclera and choroid) of the suprachoroidal space that does not exist naturally.

The implant 105 can have structural properties that cause the implant to interfere with and/or resist the natural curvature of the suprachoroidal space when implanted in the eye. In this regard, the implant 105 can have an effective or extrinsic Young's modulus (relative to the Young's modulus of the tissue boundary of the suprachbroidal space) that causes the implant to interfere with and locally change the curvature of the boundary between the sclera and the choroid when implanted in the eye. As mentioned above, the implant 105, when implanted, does not necessarily extend into a region of the suprachoroidal space that is between the sclera and the choroid. The implant can be positioned between the ciliary body and the sclera (within the supraciliary space) but still communicate with the suprachoroidal space. The implant 105 can be made of a material that has the requisite stiffness, or the implant can have structural properties, such as thickness or length, that achieve the requisite stiffness and deformation of the normal curvature of the sclera-suprachoroid boundary.

In an embodiment, the implant can be made of a material that has a Young's modulus that is less than 3,000 pounds per square inch (PSI). In another embodiment, the Young's modulus is in the range of 3,000 psi to 70,000 psi. In another embodiment, the Young's modulus is approximately 200,000 psi. In another embodiment, the Young's modulus is less than or equal to 40,000,000 psi. It should be appreciated that the aforementioned values are exemplary and non-limiting.

In an embodiment, the implant 105 can have a column strength sufficient to permit the implant 105 to be inserted into suprachoroidal space such that the distal tip of the implant 105 tunnels through the eye tissue (such as the ciliary body CB) without structural collapse or structural degradation of the implant 105. In an embodiment, the column strength can be sufficient to permit the implant to tunnel through the eye tissue into the suprachoroidal space SchS without any structural support from an additional structure such as a delivery device.

The implant 105 can be positioned in the eye so that a portion of the implant is sitting on top of the ciliary body CB. The ciliary body CB can act as a platform off of which the implant 105 can cantilever into the suprachoroidal space SchS. The implant 105 can have a relative stiffness such that, when implanted, the implant 105 deforms at least a portion of the tissue adjacent the suprachoroidal space to take on a shape that is different than the natural curvature. In this manner, the implant 105 can lift or “tent” the sclera S outward such that the suprachoroidal space SchS is formed around the distal end of the implant 105. The tenting of the sclera S as shown in FIG. 5 has been exaggerated for clarity of illustration. It should be appreciated that the actual contour of the tented region of tissue may differ in the actual anatomy. The implant 105 can act as a flow pathway between the anterior chamber AC and the suprachoroidal space SchS without blockage of the outflow pathway by surrounding tissues such as the sclera or the choroid.

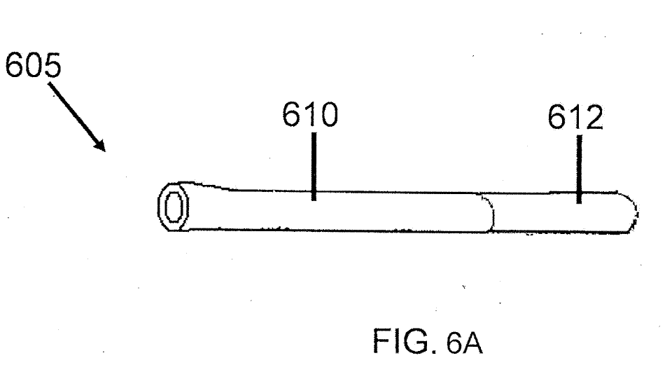

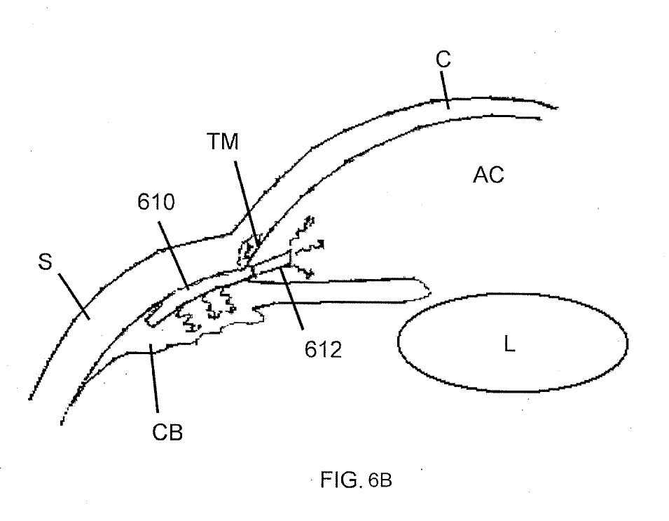

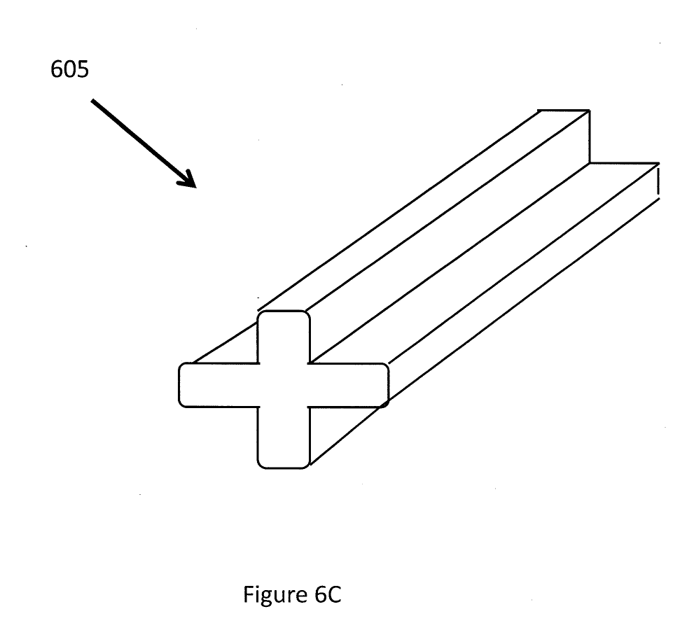

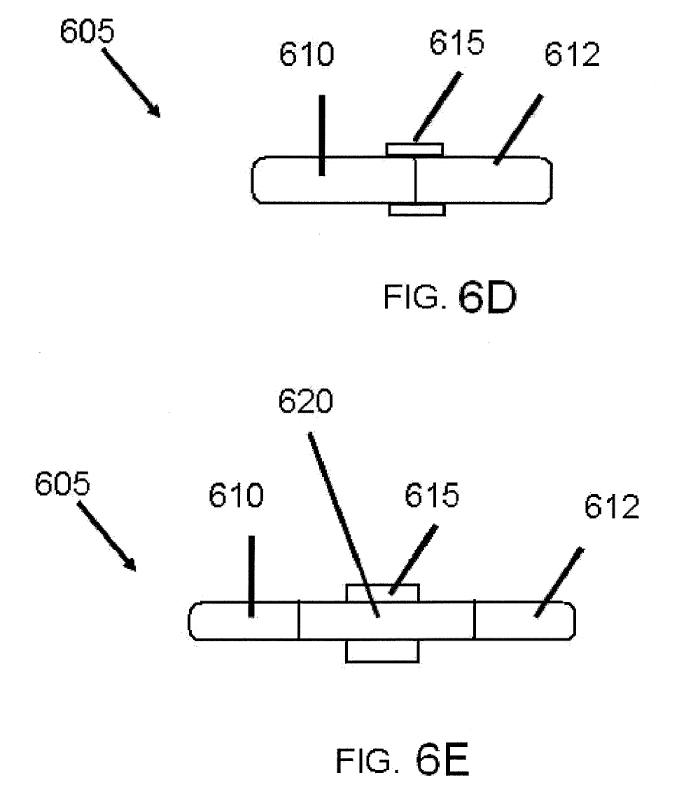

The implants described herein can be coated on an inner or outer surface with one or more drugs or other materials such as a biodegradable drug-eluting polymers impregnated with a drug, wherein the drug or material can also be used for disease treatment such as reduction of aqueous production or improved outflow of aqueous through uveoscleral structures. FIGS. 6A-6E show embodiments of drug delivery implants 605 having one or more drug delivery zones that function to control glaucoma.

The implant 605 can have a variety of configurations. For example, the implant 605 can be an elongated tubular member having a proximal end, a distal end, and a structure that permits fluid (such as aqueous humor) to flow along the length of the implant such as through or around the implant from the anterior chamber. The implant 605 can be a solid bar without an internal cavity that allows for flow of aqueous humor along an outside surface. The implant 605 can have at least one internal lumen having at least one opening for ingress of fluid and at least one opening for egress of fluid. The implant 605 can have a variety of cross-sections and shapes. For example, the implant can have a circular, oval or rectangular shape and can vary in cross-sectional shape moving along its length. In an embodiment, the implant 605 can have a star or cross-shape such that aqueous from the anterior chamber flows through one or more convoluted outer surface of the implant (see FIG. 6C). The implant can also have a braided or woven structure such as a stent.

FIG. 6A shows an example of a drug delivery implant 605 having two drug delivery zones 610, 612. The implant 605 can be coated on an inner or outer surface with one or more drugs to create the one or more drug delivery zones. For example, the drug(s) in each drug delivery zone can be embedded in a polymer (nonabsorbable or bioabsorbable) or polymer matrix that is coated on the implant such that the dispersed drug diffuses from the polymer matrix. The implant 605 can have a solid structure with cut-outs or include a braided portion such that the openings are spanned by a polymer matrix with drug dispersed throughout. The implant 605 can also include a surface layer of materials, for example PTFE, polyimide, Hydrogel, heparin, therapeutic drugs such as anti-glaucoma drugs or antibiotics. Layers and coatings of the implants can be a biocompatible drug-polymer blend suitable for ocular and intra-ocular drug delivery having suitable release kinetics. For example, the implant can be coated with a polymeric film containing a therapeutic that permits delivery of a quantity of the therapeutic to the surrounding tissues over a period of time and according to particular diffusion kinetics. Alternatively, the implant 605 can have one or more internal reservoirs (not shown) associated with each of the drug delivery zones that fluidically communicate with the surface of the implant 605 such that the drugs elute therefrom and come into contact with adjacent tissues. Drugs from the drug delivery zones can elute to the surface of the implant 605 through openings extending from the internal surface to the external surface. The reservoirs can be refillable and/or a single-use reservoir.

Drug-polymer blends can exhibit multi-phasic drug release profiles, which can include an initial burst of drug and a period of sustained drug release as is known in the art. The release profile can be manipulated such as by adjusting features of the composition like polymer(s), drug(s), level of drug loading, surface area and dimensions of the implant etc. The initial burst can be shortened by removing or rinsing the blend of drug at or near the surface of the implant or by coating the composition with a bioerodible polymer that can be drug free or have a reduced drug content. The implant can be coated or loaded with a slow-release substance to have prolonged effects on local tissue surrounding the implant. The slow release delivery can be designed such that the drug or other substance is released over a desired duration as is known in the art. The implant can be made of a material that is admixed with a substance for slow-release into the surrounding tissues. The implant can also include small reservoir(s) of drug that can be opened such as by a laser or other energy source to apply a small electrical voltage to release the desired dose of the drug(s) on demand.

The coatings can be spray-coated, dip coated, printed, or otherwise deposited as is known in the art. The coating can be uniform or non-uniform such as dots or stripes or other pattern of material. The implant can include one or more layers of the coating. For example, a first or base layer can provide adhesion, a main layer can hold the drug to be eluted and a top coat can be used to slow down the release of the drug and extend its effect. The implant can surround a core of drug that can be released through openings in the structure of the implant. The implants can be prepared by simultaneously dissolving the polymer, drug, and, if present, optional component(s) in an organic solvent system capable of forming a homogenous solution of the polymer, drug, and optional component(s), solvent-casting the solution and then evaporating the solvent to leave behind a uniform, homogenous blend of polymer, drug and optional component(s). The drug-polymer matrices can be fabricated by known methods (e.g., fiber spinning, electro-spinning, solvent casting, injection molding, thermoforming, etc.,) to produce a desired structure for the implant. Depending on the thermal stability of the drug and the polymer, the articles can be shaped by conventional polymer-forming techniques such as extrusion, sheet extrusion, blown film extrusion, compression molding, injection molding, thermoforming, spray drying, injectable particle or microsphere suspension, and the like to form drug delivery devices.

The polymeric material for the implant, coatings or films used herein can vary including, but not limited to block copolymers, poly(vinyl aromatic) block, polystyrene block, a poly(alpha methyl styrene) block, a polyolefin block, polyisobutylene block, a polybutadiene block, a polyisoprene block and a polybutene block, polystyrene-polyisobutylene-polystyrene triblock copolymer, poly(lactic acid), polyethylene-vinyl acetate, poly(lactic-co-glycolic acid), poly(L-lactide), poly(D,L-lactide), poly(D,L-lactide-co-trimethylene carbonate), collagen, heparinized collagen, poly(caprolactone), poly(glycolic acid), poly butyl methacrylic, poly(alpha-hydroxy acid), or copolymer comprising an olefin monomer. Other biocompatible materials can include, but are not limited to polyvinyl alcohol, polyvinyl pyrolidone, polytetrafluoroethylene, expanded polytetrafluoroethylene, fluorinated polymer, fluorinated elastomer, flexible fused silica, polyolefin, polyester, polysilicon, and/or a mixture of the aforementioned biocompatible materials, and the like.

Although the implants are shown herein as including two drug delivery zones, it should be appreciated that one, two, three, or more drug delivery zones can be included on each implant. Further, each drug delivery zone can deliver one or more drugs. The implants described herein can be removed from the eye upon completion of a drug delivery protocol. Alternatively, the implants can be biodegradable and need not be removed from the eye after administration of the drug protocol.

As mentioned, the drug delivery zones can be formulated depending on where the zone is oriented within the eye upon implantation of the device. Orientation of the drug delivery zones with respect to the adjacent tissues can be selected based on where drug delivery is desired. For example, drugs that affect outflow of aqueous, for example through the trabecular meshwork TM can be embedded or delivered from a drug delivery zone positioned in the anterior chamber, near the trabecular meshwork, iris, Schlemm's canal and the like. Drugs that affect production of aqueous from epithelial cells of the ciliary body CB can be can be embedded or delivered from a drug delivery zone positioned near the ciliary body, the epithelial cells of the ciliary body, the boundary between the ciliary body and the sclera, the suprachoroidal space and the like.

The implant can have one or more drug delivery zones 610, 612 (see FIG. 6A). The implant 605 can be implanted such that one drug delivery zone 610 is positioned in a first anatomical location, for example between the ciliary body CB and the sclera S, and the other drug delivery zone 612 is positioned in a second anatomical location, such as within the anterior chamber (see FIG. 6B). The type of drug delivered from each drug delivery zone can be tailored to where in the eye anatomy the drug delivery zone is positioned. Zone 610 positioned between the ciliary body CB and the sclera S can contain drug(s) that affect the ciliary body, for example, a drug that acts on the ciliary body epithelial cells to decrease aqueous humor production. The second drug delivery zone 612 can protrude into the anterior chamber AC. This zone 612 can contain drugs that increase outflow of aqueous humor. The drugs eluting from zone 612 can enter the aqueous near the iris and increase outflow through trabecular meshwork TM by known drug mechanisms. This tailored formulation of the drug delivery zones allows for a direct route of administration to intended drug targets within the eye. Drug dosage can be reduced compared to, for example, systemic delivery or for avoiding problems with wash-out. The implant as well as each drug delivery zone relative to the implant can have a length that is suitable for desired delivery of a drug in and around various structures within the eye.

Further, it can also be advantageous to separate the drug delivery zones 610, 612 on the implant 605 with a non-drug delivery zone, a swelling component and/or a sealing barrier and the like. In the embodiment shown in FIG. 6D, the implant 605 includes one or more expandable components 615 that can swell and seat the implant within the tissue dissection channel. The drugs eluting from each drug delivery zone 610, 612 can be kept separate from one another. The external expandable component 615 can also prevent aqueous outflow through the dissection channel such as around the outside surface of the implant 605 to prevent excessive outflow of aqueous and the problems of hypotony.

FIG. 6E shows an example of such an embodiment that has a first drug delivery zone 610 and a second drug delivery zone 612 separated by a non-drug delivery zone 620. The implant can be used to deliver one kind of drug to one structure and a second drug to a second structure without drug delivery in the non-drug delivery zone. For example, the implant can deliver a first drug to the ciliary body or the ciliary body/scleral boundary or to the suprachoroidal space. The implant can also deliver another kind of drug to the anterior chamber, iris and/or trabecular meshwork area.

As mentioned above, the implant 105 can include one or more additional retaining or retention structures, such as protrusions, wings, tines, or prongs that lodge into anatomy to retain the implant in place. These retaining structures can be embedded with a drug for targeted drug delivery to a specific anatomical region, such as the anterior chamber for the reduction of aqueous humor production. The implant can have a different drug in structures located in a more distal region, for example near the posterior chamber such as to improve aqueous outflow.

A variety of drugs can be delivered using the implants described herein. The implants can deliver antiglaucoma drugs that decrease aqueous humor production including beta-blockers, carbonic anhydrase inhibitors, alpha-adrenergic agonists and the like. The implants can deliver other antiglaucoma drugs that improve aqueous humor outflow such as prostaglandins, prostaglandin analogues, muscarinics, pilocarpine, epinephrine, and carbachol. Alpha2-adrenergic agonists such as brimonidine are thought to work by a dual mechanism of decreasing aqueous production and increasing aqueous outflow. It should be appreciated that the implant can be used to deliver other therapeutic agents, such as a steroid, an antibiotic, an anti-inflammatory agent, an anti-coagulant, an anti-proliferative, imidazole antiproliferative agent, a quinoxaline, a phsophonylmethoxyalkyl nucleotide analog, a potassium channel blocker, and/or a synthetic oligonucleotide, 5-[1-hydroxy-2-[2-(2-methoxyphenoxyl)ethylamino]ethyl]-2-methylbenzenesulfonamide, a guanylate cyclase inhibitor, such as methylene blue, butylated hydroxyanisole, and/or N-methylhydroxylamine, 2-(4-methylaminobutoxy)diphenylmethane, apraclonidine, timolol, a cloprostenol analog or a fluprostenol analog, a crosslinked carboxy-containing polymer, a sugar, and water, a non-corneotoxic serine-threonine kinase inhibitor, a nonsteroidal glucocorticoid antagonist, miotics (e.g., pilocarpine, carbachol, and acetylcholinesterase inhibitors), sympathomimetics (e.g., epinephrine and dipivalylepinephxine), beta-blockers (e.g., betaxolol, levobunolol and timolol), carbonic anhydrase inhibitors (e.g., acetazolamide, methazolamide and ethoxzolamide), and prostaglandins (e.g., metabolite derivatives of arachindonic acid, or any combination thereof.

It should be appreciated that other ocular conditions besides glaucoma can be treated with the drug delivery implants described herein. For example, the implants can deliver drugs for the treatment of retinal disease, proliferative vitreoretinopathy, diabetic retinopathy, uveitis, keratitis, cytomegalovirus retinitis, cystoid macular edema, herpes simplex viral and adenoviral infections. It also should be appreciated that medical conditions besides ocular conditions can be treated with the drug delivery implants described herein. For example, the implants can deliver drugs for the treatment of inflammation, infection, cancerous growth.

More than one disease or condition can be treated from one implant. For example, both retinal disease and glaucoma can be treated from one implant bar. It should also be appreciated that more than two or three drug delivery zones are considered herein and that different medications can be used to treat different portions of the eye in the different zones of the implant.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

What is claimed is:

1. An ocular implant for treating an eye, comprising:
an elongate member having a proximal end with at least one inflow port, a distal end with at least one outflow port, and a longitudinal, internal lumen extending through the elongate member, wherein the distal end of the elongate member is in fluid communication with the suprachoroidal space such that the proximal end of the elongate member remains in fluid communication with the anterior chamber when the elongate member is implanted in the eye; and
at least one polymeric film surrounding at least a portion of the elongate member, the film comprising a first drug delivery zone embedded with a first drug, wherein the first drug diffuses from the polymeric film over time into the eye at a first anatomical location.

2. The implant of claim 1, wherein the polymeric film further comprises a second drug delivery zone embedded with a second drug and the second drug diffuses from the polymeric film into the eye over time at a second anatomical location.

3. The implant of claim 1, wherein the first anatomical location comprises the ciliary body, epithelial cells of the ciliary body, the boundary between ciliary body and sclera, or the suprachoroidal space.

4. The implant of claim 3, wherein the first drug reduces aqueous humor production or inflow of aqueous humor to the anterior chamber.

5. The implant of claim 4, wherein the first drug is selected from the group comprising a carbonic anhydrase inhibitor, beta blocker, and an alpha-agonist or a combination thereof.

6. The implant of claim 2, wherein the second anatomical location comprises the anterior chamber, iris, or trabecular meshwork.

7. The implant of claim 6, wherein the second drug increases outflow of aqueous humor from the anterior chamber.

8. The implant of claim 7, wherein the second drug is selected from the group comprising a prostaglandin, prostaglandin analogue, muscarinic, pilocarpine, carbachol, alpha 2 adrenergic agonist, and epinephrine, or a combination thereof.

9. The implant of claim 1, wherein the polymeric film comprises a biocompatible material selected from the group consisting of poly(lactic acid), polyethylene-vinyl acetate, polybutyl methacrylic, poly(lactic-co-glycolic acid), poly(D,L-lactide), poly(D,L-lactide-co-trimethylene carbonate), collagen, heparinized collagen, poly(caprolactone), poly(glycolic acid), a copolymer and a combination thereof.

10. The implant of claim 2, wherein the first drug and the second drug are the same drug.

11. The implant of claim 2, wherein the first drug and the second drug are not the same drug.

12. The implant of claim 1, further comprising a second polymeric film surrounding at least a portion of the first polymeric film.

13. The implant of claim 12, wherein the second polymeric film alters a parameter of diffusion kinetics of the first drug from the first drug delivery zone.
